(19) United States
(12) Patent Application Publication
(10) Pub. No.: US 2013/0130991 A1
(43) Pub. Date: May 23, 2013

(54) USE OF A PEPTIDE ENHANCING THE ABILITY OF RADIATION THERAPY TO KILL CANCER CELLS

(71) Applicants: Christian Widmann, Lausanne (CH); Oscar Matzinger, Grandvaux (CH)

(72) Inventors: Christian Widmann, Lausanne (CH); Oscar Matzinger, Grandvaux (CH)

(73) Assignee: UNIVERSITE DE LAUSANNE, Lausanne (CH)

(21) Appl. No.: 13/813,702

(22) PCT Filed: Jul. 29, 2011
(86) PCT No.: PCT/EP2011/063078

§ 371 Date: Feb. 1, 2013

Related U.S. Application Data
(60) Provisional application No. 61/344,475, filed on Aug. 2, 2010.

(51) Int. Cl. A61K 41/00

(52) U.S. Cl. CPC A61K 41/0038
USPC 514/19.3; 514/19.2

(57) ABSTRACT

The present invention relates to a peptide consisting essentially of the N2 sequence of the RasGAP protein, a bio logically active fragment thereof, or a variant thereof, which is useful for the preparation of a medicament for the treatment of cancer. Furthermore, it relates to a method of treatment of cancer comprising administering to a subject in need thereof, a therapeutically effective amount of the peptide of the invention.

### FIELD OF THE INVENTION

The present invention relates to a peptide useful for the preparation of a medicament for the treatment of cancer. Furthermore, it relates to a method of treatment of cancer comprising administering to a subject in need thereof, a therapeutically effective amount of the peptide of the invention.

### BACKGROUND OF THE INVENTION

Since the discovery of radium by the Curies, radiotherapy has offered incalculable benefits for cancer patients. It is presumed that the essential target for radiation is cellular DNA where it acts through the formation of free radicals to directly or indirectly cause double-stranded breaks. It is these double-stranded breaks in the DNA that are traditionally felt to be the lethal lesion that malignant cells sustain from therapeutic radiation.

Radiation is used on a wide variety of tumors for both curative and palliative indications. The ability to target radiotherapy and avoid normal tissue outside the planned radiotherapy field has been dramatically improved with the development of conformal radiotherapy and intensity-modulated radiation therapy. However, despite these advances, radiation toxicity remains a major obstacle to effective therapy and the dose of radiotherapy that can be administered to tumors is often limited by the toxic effects of the therapy on healthy tissue.

Therapeutic gain is defined by an increase in tumor control probability (and hence survival) without a parallel increase in the severity of side effects. In an ideal setting, the probability of normal tissue damage should be minimal at a dose level that induces maximal probability of tumor control. Several strategies of combined modality treatments have been developed in order to improve the therapeutic index. Most of these strategies combine chemotherapeutic agents with radiotherapy and are considered as standard treatments in many tumor entities (head & neck; cervix; lung; gastro intestinal; brain . . . ).

However as the prognosis of many cancers remains poor there is a need to develop new strategies to improve tumor control by radiation.

### SUMMARY OF THE INVENTION

This object has been achieved by providing the use of a peptide consisting essentially of the N2 sequence of the RasGAP protein, a biologically active fragment thereof, or a variant thereof, for the preparation of a medicament for the treatment of cancer and/or tumors in a patient in need thereof.

A further object of the present invention is to provide a peptide consisting essentially of the N2 sequence of the RasGAP protein, a biologically active fragment thereof, or a variant thereof, for the treatment of cancer and/or tumors in a patient in need thereof wherein said peptide, or a biologically active fragment thereof, or a variant thereof, is administered prior to, during and/or after said patient was subjected to a radiation therapy and wherein said peptide, or a biologically active fragment thereof, or a variant thereof, enhances the ability of said radiation therapy to kill cancer cells or to enhance the effect of said radiation therapy on tumors.

Furthermore, the invention provides a method of treatment of cancer and/or tumors comprising administering to a patient in need thereof, a therapeutically effective amount of i) a peptide consisting essentially of the N2 sequence of the RasGAP protein, a biologically active fragment thereof, or a variant thereof, or ii) a peptide consisting essentially of the N2 sequence of the RasGAP protein, a biologically active fragment thereof, or a variant thereof, conjugated to an agent which increases the accumulation of said peptide in a cell, prior to, during and/or after said patient was subjected to a radiation therapy and so that said peptide enhances the ability of said radiation therapy to kill cancer cells or reduce tumors.

The invention further provides an in vivo method of sensitizing cancer cells to radiation therapy comprising contacting a cell with at least one peptide consisting essentially of the N2 sequence of the RasGAP protein, a biologically active fragment thereof, or a variant thereof, conjugated or not to an agent which increases the accumulation of said peptide in said cell.

Also provided is an in vivo method of enhancing radiation therapy in a patient in need thereof, said method comprising contacting a cell with at least one peptide consisting essentially of the N2 sequence of the RasGAP protein, a biologically active fragment thereof, or a variant thereof, conjugated or not to an agent which increases the accumulation of said peptide in said cell wherein said peptide is contacted prior to, during and/or after said patient was subjected to a radiation therapy.

### BRIEF DESCRIPTION OF THE FIGURES

FIG. 1 shows the effects of irradiation on tumors cells in the presence or in the absence of TAT-RasGAP₃₁₇₋₃₂₆. Figure A: Hela cells were exposed to the indicated doses of γ-irradiation in the presence or in absence of 20 μM TAT-RasGAP317-326. Apoptosis was determined by scoring the percentage of cells with pycnotic nuclei. Figure B: The indicated cell lines were treated with increasing doses of γ-irradiation in the presence or in the absence of TAT or 20 μM TAT-RasGAP317-326. After two weeks, the number of colonies was determined.

FIG. 2 is a schematic representation of the different constructs used in this study. SH means Src homology domain.

FIG. 3 shows the radio-sensitizing effect of TAT-RasGAP317-326 on the non-tumorigenic HaCAT cells cell line (3B) and the radio-sensitizing effect of TAT-RasGAP317-326 on the tumor volume of mice bearing wild-type HCT116 xenografts (3C). HCT116 p53 knock-out cells (3A,) and non-tumorigenic HaCAT cells (3B) were subjected to the indicated doses of γ-irradiation in the presence or in absence of 20 μM TAT-RasGAP317-326. A CFA was then performed and two weeks later, the number of colonies was determined. FIG. 3C represents the tumor volume of mice bearing wild-type HCT116 xenografts in 4 treatment groups of 3 mice each.

### DETAILED DESCRIPTION OF THE INVENTION

The present invention relates to the use of a peptide consisting essentially of the N2 sequence of the RasGAP protein, a biologically active fragment thereof, or a variant thereof, for the preparation of a medicament for the treatment of cancer and/or tumors in a patient in need thereof wherein the medicament is administered prior to, during and/or after said patient was subjected to a radiation therapy and wherein said medicament enhances the ability of said radiation therapy to kill cancer cells or to enhance the effect of said radiation therapy on tumors.

The present invention also relates to a peptide consisting essentially of the N2 sequence of the RasGAP protein, a biologically active fragment thereof, or a variant thereof, for the treatment of cancer and/or tumors in a patient in need thereof wherein said peptide, or a biologically active fragment thereof, or a variant thereof, is administered prior to, during and/or after said patient was subjected to a radiation therapy and wherein said peptide, or a biologically active fragment thereof, or a variant thereof, enhances the ability of said radiation therapy to kill cancer cells or to enhance the effect of said radiation therapy on tumors.

For the ease of reading, the phrase “a/the peptide of the invention” used throughout the description refers to “a peptide consisting essentially of the N2 sequence of the RasGAP protein, a biologically active fragment thereof, or a variant thereof”.

As used herein, the terms “peptide”, “protein”, “polypeptide”, “polypeptidic” and “peptidic” are used interchangeably to designate a series of amino acid residues connected to the other by peptide bonds between the alpha-amino and carboxy groups of adjacent residues.

The term “comprise” or “comprising” is generally used in the sense of include/including, that is to say permitting the presence of one or more features or components. Additionally, the term “comprising” also encompasses the term “consisting”.

The term “cancer” refers to or describes the physiological condition in mammals that is typically characterized by unregulated cell growth. Non-limiting examples of cancers are selected among the group comprising all kind of cancers and tumors arising from:
Connective Tissue: such as Fibrosarcoma, Myxosarcoma, Liposarcoma, Chondrosarcoma, Osteosarcoma, Chordoma, Malignant fibrous histiocytoma
Endothelium and Mesothelium: such as Hemangiosarcoma, angiosarcoma, Lymphangiosarcoma, Mesothelioma
Blood and Lymphoid Cells: such as Leukemia, of various types; aleukemic leukemia, Plasmacytoma; multiple myeloma; Hodgkin lymphoma and Non-Hodgkin lymphoma
Muscle: such as Leiomyosarcoma, Rhabdomyosarcoma and
Epithelial Tissues: such as Squamous cell carcinoma; epidermoid carcinoma, malignant skin adnexal tumors, Adenocarcinoma, hepatocellular carcinoma, Renal cell carcinoma; hypernephroma, Cholangiocarcinoma, Transitional cell carcinoma, Choriocarcinoma, Seminoma; embryonal cell.

By “cancer cell” is meant a cell arising in an animal in vivo which is capable of undesired and unregulated cell growth or abnormal persistence or abnormal invasion of tissues. In vitro this term also refers to a cell line that is a permanently immortalized established cell culture that will proliferate indefinitely and in an unregulated manner given appropriate fresh medium and space.

The term “tumor” as used herein, refers to an abnormal mass of tissue that results from excessive cell division.

“Radiation therapy” refers to the use of high-energy radiation to shrink tumors and kill cancer cells. Examples of radiation therapy include, without limitation, external radiation therapy and internal radiation therapy (also called brachytherapy).

External radiation therapy is most common and typically involves directing a beam of direct or indirect ionizing radiation to a tumor or cancer site. While the beams of radiation, the photons, the Cobalt or the particle therapy are focused to the tumor or cancer site, it is nearly impossible to avoid exposure of normal, healthy tissue. Energy source for external radiation therapy is selected from the group comprising direct or indirect ionizing radiation (for example: x-rays, gamma rays and particle beams or combination thereof).

Internal radiation therapy involves implanting a radiation-emitting source, such as beads, wires, pellets, capsules, etc., inside the body, at, or near to the tumor site. Energy source for internal radiation therapy is selected from the group of radioactive isotopes comprising: iodine (iodine-125 or iodine-131), strontium89, radioisotopes of phosphorous, palladium, cesium, indium, phosphate, or cobalt, and combination thereof. Such implants can be removed following treatment, or left in the body inactive. Types of internal radiation therapy include, but are not limited to, interstitial, and intracavity brachytherapy (high dose rate, low dose rate, pulsed dose rate).

A currently less common form of internal radiation therapy involves biological carriers of radioisotopes, such as with radio-immunotherapy wherein tumor-specific antibodies bound to radioactive material are administered to a patient. The antibodies bind tumor antigens, thereby effectively administering a dose of radiation to the relevant tissue.

Methods of administering radiation therapy are well known to those of skill in the art.

“RasGAP”, a regulator of Ras and Rho GTP-binding proteins, is an unconventional caspase substrate because it can induce both anti- and pro-apoptotic signals, depending on the extent of its cleavage by caspases. At low levels of caspases, RasGAP is cleaved at position 455, generating an N-terminal fragment (fragment N, of about 56 kD) and a C-terminal fragment (fragment C, of about 64 kD). Fragment N appears to be a general blocker of apoptosis downstream of caspase activation (Yang J.-Y. and Widmann C., Mol. Cell. Biol., 21, 5346, 2001 and J. Biol. Chem., 277, 14641, 2002b). At high levels of caspase activity, fragment N is further cleaved at position 157 thus generating two fragments, N1 (amino acids 1 to 157) and N2 (corresponding to amino acids 158-455 in the human RasGAP protein sequence).

Recently, the Applicant of the present invention has described a cell permeable form of a peptide derived from the N2 sequence of the RasGAP protein (TAT-RasGAP₃₁₇₋₃₂₆) that specifically sensitizes tumor cells to genotoxin-induced death (Michod D, Yang J Y, Chen J, Bonny C, Widmann C (2004) A RasGAP-derived cell permeable peptide potently enhances genotoxin-induced cytotoxicity in tumor cells. Oncogene 23: 8971-8978). The peptide does not by itself modulate apoptosis of tumor cells, nor does it sensitize non-tumor cells to genotoxin-induced apoptosis (Michod et al., 2004, see above). This peptide increases the ability of genotoxins to promote cytochrome c release from the mitochondria via the p53-PUMA pathway (Michod D, Widmann C (2007) TAT-RasGAP₃₁₇₋₃₂₆ requires p53 and PUMA to sensitize tumor cells to genotoxins. Mol. Cancer Res. 5: 497-507). This increased cytochrome c release results in stronger caspase activation and augmented cell death (Michod D, Widmann C, 2007, see above). Importantly, this peptide increases the ability of cisplatin and other genotoxins to inhibit tumor growth. Doses that are at >100 fold below the lethal doses (the LD50 is between ˜2.5 and ˜5.0 mg/kg) still exert a genotoxin-sensitization on tumor growth (Michod, D., Annibaldi, A., Schaefer, S., Dapples, C., Rochat, B., and Widmann, C. (2009). Effect of RasGAP N2 fragment-derived peptide on tumor growth in mice. J. Natl. Cancer Inst. 101, 828-832).

Surprisingly, the Applicants of the present invention have shown that the same peptide, i.e. a peptide derived from the N2 sequence of the RasGAP protein also enhances the efficacy of irradiation-mediated cell killing in different cell lines as assessed by apoptosis and clonogenic tests.

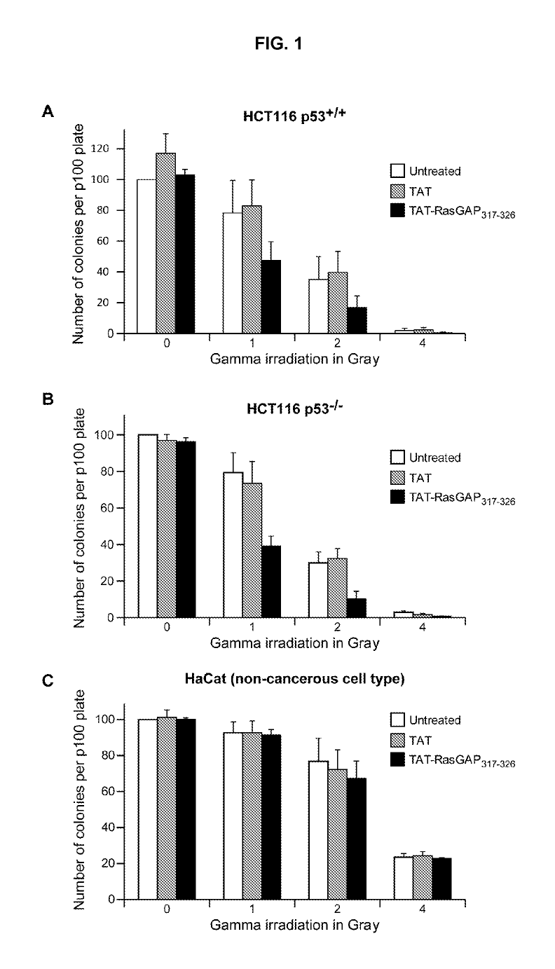

In contrast to what has been reported before for the genotoxin-sensitization on tumor growth (Michod D, Widmann C, 2007, see above), the peptide of the invention does not require a functional p53 cellular status to increase the sensitivity of cancer and tumor cells to radiotherapy. FIG. 1B shows that a peptide derived from the N2 sequence of the RasGAP protein (TAT-RasGAP₃₁₇₋₃₂₆) increases the efficacy of γ-irradiation on tumor cells, regardless whether the tumor cells bear a functional p53 gene or not.

The term “enhancing” as used herein refers to the capacity of the peptide of the invention to increase the effect of radiation therapy to kill cancer cells or tumors. This capacity can be measured in vitro by, for example, clonogenic assay or by measuring the percentage of apoptosis of cells treated with the peptide and incubated with at least one drug by scoring the number of cells displaying pycnotic nuclei (a marker of apoptotic cells). Typically, the results are compared to those from radiotherapy treated cells that were not treated with said peptide. A peptide that leads to a statistically significant increase of apoptosis in cells at a given concentration or that decreases in a statistically significant manner the dose of the radiotherapy to induce a given response, will be considered as enhancing the ability of said radiation therapy to kill cancer cells or to enhance the effect of said radiation therapy on tumors. For example, the peptide of the invention may decrease the dose of the radiotherapy to induce a given response by, 2% or more, 3% or more, 4% or more 5% or more, such as by 10% or more, such as by 20% or more, such as by 30% or more, such as by 50% or more, such as by 90% or more, such as 95% or more, as compared to a suitable control.

In other embodiments, the peptide of the invention may increase specifically the apoptosis in cancer cells at a given concentration. For example, methods of the invention may increase the rate of apoptosis in cancer cells by 2% or more, such as by 5% or more, such as by 10% or more, such as by 25% or more, such as by 50% or more, such as by 75% or more, such as by 100% or more, such as by 200% or more, including by 500% or more, as compared to a suitable control.

In other embodiments, the peptide of the invention may significantly reduce the size or volume of the tumor by, 2% or more, 3% or more, 4% or more 5% or more, such as by 10% or more, such as by 20% or more, such as by 30% or more, such as by 50% or more, such as by 90% or more, such as 95% or more, as compared to a suitable control.

Furthermore, there are evidences showing that the enhancing properties selectively in cancer cells, i.e. specific to these cells.

As used herein, by the term “selectively” is meant that the peptide of the invention enhances the ability of the radiation therapy to kill cells, or enhances the effect of said radiation therapy on tumors, at a given concentration, specifically in cancer cells but importantly not in non cancer cells.

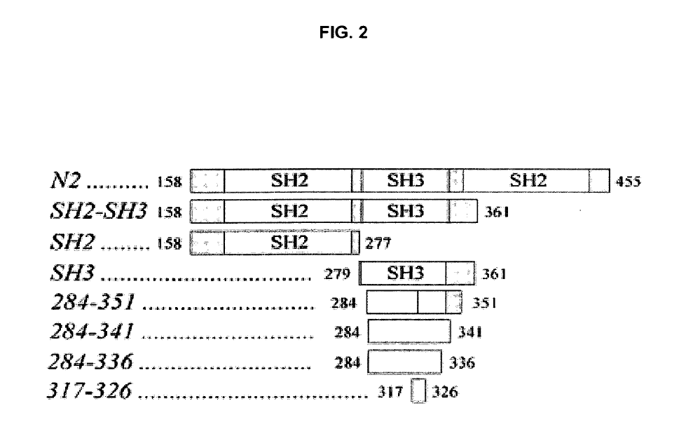

The N2 sequence of the RasGAP protein, when derived from human, refers to a 36 kD protein consisting of 297 amino acids which encompasses two SH2 and one SH3 domain as shown in FIG. 2. In general, Src homology 2 (SH2) domains are involved in recognition of phosphorylated tyrosine whereas Src homology 3 (SH3) domains are often indicative of a protein involved in signal transduction. The amino acid sequence of the human RasGAP protein is as set forth in SEQ ID No 6:

SLDGPEYEEEEVAIPLTAPPTNQWYHGKLDRTIAEERLRQAGKSGSYLIRESD
RRPGSFVLSFLSQMNVVNHFRIIAMCGDYYIGGRRFSSLSDLIGYYSHVSCLLKGEKLLYPVAPPEPVED
RRRVRAILPYTKVPDTDEISFLKGDMFIVHNELEDGWMWVTNLRTDEQGLIVEDLVEEVGREEDPHEGKI
WFHGKISKQEAYNLLMTVGQVCSFLVRPSDNTPGDYSLYFRTNENIQRFKICPTPNNQFMMGGRYYNSIG
DIIDHYRKEQIVEGYYLKEPVPMQDQEQVLNDTVD

“A biologically active fragment of the N2 sequence of the RasGAP protein” refers to a sequence containing less amino acids in length than the N2 sequence of the RasGAP protein. This sequence can be used as long as it exhibits the same properties as the native sequence from which it derives, i.e. to enhance the ability of a radiation therapy to kill cancer cells or to enhance the effect of said radiation therapy on tumors Preferably this sequence contains less than 90%, preferably less than 60%, in particular less than 30% amino acids in length than the respective N2 sequence of the RasGAP protein.

The biologically active fragment the SH3 domain, or the variant thereof, contains preferably less than or equal to 70, more preferably less than or equal to 30, most preferably less than or equal to 10 amino acids of the amino acid sequence of the SH3 domain.

Preferably, the biologically active fragment of the N2 sequence of the RasGAP protein comprises the amino acid sequence of the SH3 domain of the N2 sequence, a part thereof, or a variant thereof.

The present invention also includes the use of a variant of the N2 sequence of the RasGAP protein or of a biologically active fragment of said variant. The term “variant” refers to a peptide having an amino acid sequence that differ to some extent from a native sequence peptide, that is an amino acid sequence that vary from the native sequence by conservative amino acid substitutions, whereby one or more amino acids are substituted by another with same characteristics and conformational roles. The amino acid sequence variants possess substitutions, deletions, and/or insertions at certain positions within the amino acid sequence of the native amino acid sequence. Conservative amino acid substitutions are herein defined as exchanges within one of the following five groups:

I. Small aliphatic, nonpolar or slightly polar residues: Ala, Ser, Thr, Pro, Gly
II. Polar, positively charged residues: His, Arg, Lys
III. Polar, negatively charged residues: and their amides: Asp, Asn, Glu, Gln
IV. Large, aromatic residues: Phe, Tyr, Trp
V. Large, aliphatic, nonpolar residues: Met, Leu, Ile, Val, Cys.
Examples of Variants are given throughout the description.

The N2 sequence, as well as a biologically active fragment and a variant thereof can be prepared by a variety of methods and techniques known in the art such as for example chemical synthesis or recombinant techniques as described in Maniatis et al. 1982, Molecular Cloning, A laboratory Manual, Cold Spring Harbor Laboratory.

The Applicants have then generated progressive truncations in the SH3 domain in an attempt to identify a minimal biologically active sequence. All these constructs or parts of the N2 sequence (FIG. 2), including the shortest one (317-326) that codes for a 10 amino acid long peptide, still enhances the ability of a radiation therapy to kill cancer cells or enhances the effect of said radiation therapy on tumors. These results show that the biological property of fragment N2 does not require a complete SH3 domain but can be mediated by a part of the SH3 domain such as a short peptidic sequence.

In particular, encompassed by the present invention, is a biologically active fragment of the SH3 domain which consists in the amino acid sequences encoded by the DNA sequences of Table 1:

TABLE 1

| DNA | | | Amino acid |
|---|---|---|---|
| Sequence | | | sequences |
| ID | Name | DNA sequences | (SEQ ID N^{o}) |
| SEQ ID N^{o} 1 | RaSGAP₂₈₄₋₃₅₁ | gaagatagaaggcgtgtacgagctattctacctta | EDRRRVRAILPYTKV |
| | | cacaaaagtaccagacactgatgaaataagtttct | PDTDEISFLKGDMFI |
| | | taaaaggagatatgttcattgttcataatgaatta | VHNELEDGWMWVTNL |
| | | gaagatggatggatgtgggttacaaatttaagaac | RTDEQGLIVEDLVEE |
| | | agatgaacaaggccttattgttgaagacctagtag | VGREEDPHEGKIWFH |
| | | aagaggtgggccgggaagaagatccacatgaagga | GKISKQEA |
| | | aaaatatggttccatgggaagatttccaaacagga | (SEQ ID N^{o} 14) |
| | | agct | |
| SEQ ID N^{o} 2 | RaSGAP₂₈₄₋₃₄₁ | gtacgagctattctaccttacacaaaagtaccaga | RVRAILPYTKVPDTD |
| | | cactgatgaaataagtttcttaaaaggagatatgt | EISFLKGDMFIVHNE |
| | | tcattgttcataatgaattagaagatggatggatg | LEDGWMWVTNLRTDE |
| | | tgggttacaaatttaagaacagatgaacaaggcct | QGLIVEDLVEEVGRE |
| | | tattgttgaagacctagtagaagaggtgggccggg | EDPHEGKIW |
| | | aagaagatccacatgaaggaaaaatatgg | (SEQ ID N^{o} 15) |
| SEQ ID N^{o} 3 | RaSGAP₂₈₄₋₃₃₆ | gtacgagctattctaccttacacaaaagtaccaga | RVRAILPYTKVPDTD |
| | | cactgatgaaataagtttcttaaaaggagatatgt | EISFLKGDMFIVHNE |
| | | tcattgttcataatgaattagaagatggatggatg | LEDGWMWVTNLRTDE |
| | | tgggttacaaatttaagaacagatgaacaaggcct | QGLIVEDLVEEVGR |
| | | tattgttgaagacctagtagaagaggtgggccgg | (SEQ ID N^{o} 16) |
| SEQ ID N^{o} 4 | RasGAP₃₁₇₋₃₂₆ | tggatgtgggttacaaatttaagaacagat | WMWVTNLRTD |
| | | | (SEQ ID N^{o} 5) |

In case the part of the SH3 domain of the N2 sequence is SEQ ID No 4 (RasGAP₃₁₇₋₃₂₆) then the resulting amino acid sequence encoded by said SEQ ID No 4 in human is WMWVTNLRTD. A comparison between the different species revealed that there are different amino acids, which are conserved among the species as shown in table 2.

TABLE 2

| | Amino acid | Amino acid |
|---|---|---|
| | sequences of | Sequence |
| Species | RasGAP₃₁₇₋₃₂₆ | ID |
| Human | WMWVTNLRTD | SEQ ID N^{o} 5 |
| Bos taurus | WMWVTNLRTD | SEQ ID N^{o} 5 |
| Mouse | WMWVTNLRTD | SEQ ID N^{o} 5 |
| Rattus norvegicus | WMWVTNLRTD | SEQ ID N^{o} 5 |
| Anopheles | WLWVTAHRTG | SEQ ID N^{o} 9 |
| Drosophilia | WLWVTAHRTG | SEQ ID N^{o} 9 |
| Variant 1* | WLWVSNLRTD | SEQ ID N^{o} 11 |
| Variant 2* | WMWVTNHRTD | SEQ ID N^{o} 12 |
| Alignment | WxWVTxxRTx | SEQ ID N^{o} 13 |

*Variants 1 and 2 are synthetic peptides and are not found in biological species

Conserved amino acids among the species are represented as bold underlined type residues whereas the X correspond to amino acid residues that can be changed by conservative, or non-conservative amino acid substitutions, without impairing the inventive properties of these 10 amino acid parts of the SH3 domain of N2.

These peptidic variants of this 10 amino acid part of the human SH3 domain of N2, and in particular the alignment sequence WXWVTXXRTX (SEQ ID No 5), are also encompassed by the present invention and they refer to peptides having an amino acid sequence that differ to some extent from the native sequence peptide, that is the amino acid sequence that vary from the native sequence WMWVTNLRTD by conservative or non-conservative amino acid substitutions, whereby one or more amino acid residues are substituted by another with same characteristics and conformational roles.

Preferably, the fragment comprising the amino acid sequence of the SH3 domain of the N2 sequence comprises the general amino acid sequence WXWVTXXRTX (SEQ ID No. 13), wherein X represents an amino acid. Variants of this general sequence comprise an amino acid selected from the group comprising WLWVTAHRTG (SEQ ID No 9), WLWVSNLRTD (SEQ ID No 11) and WMWVTNHRTD (SEQ ID No 12).

Preferably also the fragment comprising the amino acid sequence of the SH3 domain of the N2 sequence consists in the amino acid sequences encoded by the DNA sequences SEQ ID No. 1, SEQ ID No. 2, SEQ ID No. 3 or SEQ ID No. 4 or consists in the amino acid sequences selected from the groups comprising SEQ ID No. 14, SEQ ID No. 15, SEQ ID No. 16, or SEQ ID No. 5.

Usually, the peptide consisting essentially of the N2 sequence of the RasGAP protein, a fragment thereof, or a variant thereof as disclosed in the present invention is conjugated to an agent that increases the accumulation of the peptide in a cell.

Such an agent can be a compound which induces receptor mediated endocytosis such as for example the membrane transferrin receptor mediated endocytosis of transferrin conjugated to therapeutic drugs (Qian Z. M. et al., “Targeted drug delivery via the transferrin receptor-mediated endocytosis pathway” Pharmacological Reviews, 54, 561, 2002) or a cell membrane permeable carrier which can, be selected e.g. among the group of fatty acids such as decanoic acid, myristic acid and stearic acid, which have already been used for intracellular delivery of peptide inhibitors of protein kinase C (Ioannides C. G. et al., “Inhibition of IL-2 receptor induction and IL-2 production in the human leukemic cell line Jurkat by a novel peptide inhibitor of protein kinase C” Cell Immunol., 131, 242, 1990) and protein-tyrosine phosphatase (Kole H. K. et al., “A peptide-based protein-tyrosine phosphatase inhibitor specifically enhances insulin receptor function in intact cells” J. Biol. Chem. 271, 14302, 1996) or among peptides. Preferably, cell membrane permeable carriers are used, more preferably a cell membrane permeable carrier peptide is used.

In case the cell membrane permeable carrier is a peptide then it will preferably be a positively charged amino acid rich peptide.

Preferably such positively charged amino acid rich peptide is an arginine rich peptide. It has been shown in Futaki et al. (Futaki S. et al., “Arginine-rich peptides. An abundant source of membrane-permeable peptides having potential as carriers for intracellular protein delivery” J. Biol. Chem., 276, 5836, 2001), that the number of arginine residues in a cell membrane permeable carrier peptide has a significant influence on the method of internalization and that there seems to be an optimal number of arginine residues for the internalization, preferably they contain more than 6 arginines, more preferably they contain 9 arginines (R9).

The peptide of the invention may be conjugated to the cell membrane permeable carrier by a spacer. In this case the cell membrane permeable carrier is preferably a peptide.

Usually arginine rich peptides are selected from the group comprising the HIV-TAT₄₈₋₅₇ peptide, the FHV-coat₃₅₋₄₉ peptide, the HTLV-II Rex₄₋₁₆ peptide and the BMV gag₇₋₂₅ peptide. Preferably, the arginine rich peptide is HIV-TAT₄₈₋₅₇ peptide.

In case the HIV-TAT₄₈₋₅₇ peptide is conjugated to a RasGAP sequence, such as for example RasGAP₃₁₇₋₃₂₆, then two glycine residues are inserted between the TAT and RasGAP sequences as spacer to allow flexibility.

Since an inherent problem with native peptides (in L-form) is degradation by natural proteases, the peptide, as well as the cell membrane permeable peptide, of the invention may be prepared to include D-forms and/or “retro-inverso isomers” of the peptide.

In this case, retro-inverso isomers of fragments and variants of the peptide, as well as of the cell membrane permeable peptide, of the invention are prepared.

Non limiting examples of retro-inverso (R1) sequences of the peptides of the invention are selected from the group comprising the sequences listed in Table 3.

TABLE 3

| Amino acid | | |
|---|---|---|
| Sequence ID | | |
| SEQ ID N^{o} 7 | RI-TAT-RasGAP₃₁₇₋₃₂₆ | DTRLNTVWMWGGRRRQRRKKRG |
| SEQ ID N^{o} 8 | RI- RasGAP₃₁₇₋₃₂₆ | DTRLNTVWMW |
| SEQ ID N^{o} 10 | RI-Anopheles | GTRHATVWLW |
| | RasGAP₃₁₇₋₃₂₆ | |
| SEQ ID N^{o} 17 | RI-Variant 1* | DTRLNSVWLW |
| SEQ ID N^{o} 18 | RI-Variant 2* | DTRHNTVWMW |
| SEQ ID N^{o} 19 | RI-Alignment | xTRxxTVWxW |
| SEQ ID N^{o} 20 | RI- TAT -Anopheles | GTRHATVWLWGGRRRQRRKKRG |
| | RasGAP₃₁₇₋₃₂₆ | |
| SEQ ID N^{o} 21 | RI- TAT -Variant 1* | DTRLNSVWLWGGRRRQRRKKRG |
| SEQ ID N^{o} 22 | RI- TAT -Variant 2* | DTRHNTVWMWGGRRRQRRKKRG |
| SEQ ID N^{o} 23 | RI-TAT-Alignment | xTRxxTVWxWGGRRRQRRKKRG |
| SEQ ID N^{o} 24 | RI- R9-RasGAP₃₁₇₋₃₂₆ | DTRLNTVWMWGGRRRRRRRRR |
| SEQ ID N^{o} 25 | RI- R9 -Anopheles | GTRHATVWLWGGRRRRRRRRR |
| | RasGAP₃₁₇₋₃₂₆ | |
| SEQ ID N^{o} 26 | RI- R9 -Variant 1* | DTRLNTVWMWGGRRRRRRRRR |
| SEQ ID N^{o} 27 | RI- R9 -Variant 2* | DTRHNTVWMWGGRRRRRRRRR |
| SEQ ID N^{o} 28 | RI- R9 - Alignment | xTRxxTVWxWGGRRRRRRRRR |

Protecting the peptide from natural proteolysis should therefore increase the effectiveness of the specific heterobivalent or heteromultivalent compound. A higher biological activity is predicted for the retro-inverso containing peptide when compared to the non-retro-inverso containing analog owing to protection from degradation by native proteinases. Furthermore they have been shown to exhibit an increased stability and lower immunogenicity (Sela M. and Zisman E., “Different roles of D-amino acids in immune phenomena” FASEB J. 11, 449, 1997).

Retro-inverso peptides are prepared for peptides of known sequence as described for example in Sela and Zisman, (1997).

By “retro-inverso isomer” is meant an isomer of a linear peptide in which the direction of the sequence is reversed and the chirality of each amino acid residue is inverted; thus, there can be no end-group complementarity.

Also encompassed by the present invention are modifications of the peptide (which do not normally alter primary sequence), including in vivo or in vitro chemical derivitization of peptides, e.g., acetylation or carboxylation. Also included are modifications of glycosylation, e.g., those made by modifying the glycosylation patterns of a peptide during its synthesis and processing or in further processing steps, e.g., by exposing the peptide to enzymes which affect glycosylation e.g., mammalian glycosylating or deglycosylating enzymes. Also included are sequences which have phosphorylated amino acid residues, e.g., phosphotyrosine, phosphoserine, or phosphothreonine.

The invention also includes analogs in which one or more peptide bonds have been replaced with an alternative type of covalent bond (a “peptide mimetic”) which is not susceptible to cleavage by peptidases. Where proteolytic degradation of the peptides following injection into the subject is a problem, replacement of a particularly sensitive peptide bond with a noncleavable peptide mimetic will make the resulting peptide more stable and thus more useful as an active substance. Such mimetics, and methods of incorporating them into peptides, are well known in the art.

Also useful are amino-terminal blocking groups such as t-butyloxycarbonyl, acetyl, theyl, succinyl, methoxysuccinyl, suberyl, adipyl, azelayl, dansyl, benzyloxycarbonyl, fluorenylmethoxycarbonyl, methoxyazelayl, methoxyadipyl, methoxysuberyl, and 2,4,-dinitrophenyl. Blocking the charged amino- and carboxy-termini of the peptides would have the additional benefit of enhancing passage of the peptide through the hydrophobic cellular membrane and into the cell.

When recombinant techniques are employed to prepare a peptide consisting essentially of the N2 sequence of the RasGAP protein, a fragment thereof, or a variant thereof, in accordance with the present invention, nucleic acid sequences encoding the polypeptides are preferably used. With regard to the method to practise recombinant techniques, see for example, Maniatis et al. 1982, Molecular Cloning, A laboratory Manual, Cold Spring Harbor Laboratory and commercially available methods.

Accordingly the present invention also relates to a purified and isolated nucleic acid sequence encoding a peptide consisting essentially of the N2 sequence of the RasGAP protein, a fragment thereof, or a variant thereof as described above.

“A purified and isolated nucleic acid or nucleic acid sequence” refers to the state in which the nucleic acid sequence encoding the peptide of the invention, or nucleic acid encoding such peptide consisting essentially of the N2 sequence of the RasGAP protein, a fragment thereof, or a variant thereof will be, in accordance with the present invention.

A purified and isolated nucleic acid or nucleic acid sequence encompassed by the present invention might be DNA, RNA, or DNA/RNA hybrid.

DNA which can be used herein is any polydeoxynucleotide sequence, including, e.g. double-stranded DNA, single-stranded DNA, double-stranded DNA wherein one or both strands are composed of two or more fragments, double-stranded DNA wherein one or both strands have an uninterrupted phosphodiester backbone, DNA containing one or more single-stranded portion(s) and one or more double-stranded portion(s), double-stranded DNA wherein the DNA strands are fully complementary, double-stranded DNA wherein the DNA strands are only partially complementary, circular DNA, covalently-closed DNA, linear DNA, covalently cross-linked DNA, cDNA, chemically-synthesized DNA, semi-synthetic DNA, biosynthetic DNA, naturally-isolated DNA, enzyme-digested DNA, sheared DNA, labeled DNA, such as radiolabeled DNA and fluorochrome-labeled DNA, DNA containing one or more non-naturally occurring species of nucleic acid.

DNA sequences that encode a peptide consisting essentially of the N2 sequence of the RasGAP protein, a fragment thereof, or a variant thereof, can be synthesized by standard chemical techniques, for example, the phosphotriester method or via automated synthesis methods and PCR methods.

The purified and isolated DNA sequence encoding a peptide consisting essentially of the N2 sequence of the RasGAP protein, a fragment thereof, or a variant thereof, according to the invention may also be produced by enzymatic techniques. Thus, restriction enzymes, which cleave nucleic acid molecules at predefined recognition sequences can be used to isolate nucleic acid sequences from larger nucleic acid molecules containing the nucleic acid sequence, such as DNA (or RNA) that codes for a peptide consisting essentially of the N2 sequence of the RasGAP protein, a fragment thereof, or a variant thereof.

Encompassed by the present invention is also a nucleic acid in the form of a polyribonucleotide (RNA), including, e.g., single-stranded RNA, cRNA, double-stranded RNA, double-stranded RNA wherein one or both strands are composed of two or more fragments, double-stranded RNA wherein one or both strands have an uninterrupted phosphodiester backbone, RNA containing one or more single-stranded portion(s) and one or more double-stranded portion(s), double-stranded RNA wherein the RNA strands are fully complementary, double-stranded RNA wherein the RNA strands are only partially complementary, covalently crosslinked RNA, enzyme-digested RNA, sheared RNA, mRNA, chemically-synthesized RNA, semi-synthetic RNA, biosynthetic RNA, naturally-isolated RNA, labeled RNA, such as radiolabeled RNA and fluorochrome-labeled RNA, RNA containing one or more non-naturally-occurring species of nucleic acid.

Preferably used as nucleic acid is a purified and isolated DNA sequence selected from the group comprising SEQ ID No 1, SEQ ID No 2, SEQ ID No 3, or SEQ ID No 4.

The present invention also includes variants of the aforementioned sequences that are nucleotide sequences that vary from the reference sequence by conservative nucleotide substitutions, whereby one or more nucleotides are substituted by another with same characteristics.

The invention also encompasses allelic variants of the disclosed purified and isolated nucleic sequence; that is, naturally-occurring alternative forms of the isolated and purified nucleic acid that also encode peptides that are identical, homologous or related to that encoded by the purified and isolated nucleic sequences. Alternatively, non-naturally occurring variants may be produced by mutagenesis techniques or by direct synthesis.

The aforementioned purified and isolated nucleic acid sequence encoding a peptide consisting essentially of the N2 sequence of the RasGAP protein, a fragment thereof, or a variant thereof, may further comprise a nucleotide sequence encoding a cell membrane permeable carrier peptide.

Yet another concern of the present invention is to provide an expression vector comprising at least one copy of the isolated and purified nucleic acid sequence encoding a peptide consisting essentially of the N2 sequence of the RasGAP protein, a fragment thereof, or a variant thereof as described above. Preferably the isolated and purified nucleic acid sequence encoding a peptide of the invention is DNA.

As used herein, “vector”, “plasmid” and “expression vector” are used interchangeably, as the plasmid is the most commonly used vector form.

The vector may further comprise a nucleotide sequence encoding a cell membrane permeable carrier peptide in accordance with the invention. The choice of an expression vector depends directly, as it is well known in the art, on the desired functional properties, e.g., peptide expression and the host cell to be transformed or transfected.

Additionally, the expression vector may further comprise a promoter operably linked to the purified and isolated DNA sequence. This means that the linked isolated and purified DNA sequence encoding the peptide of the present invention is under control of a suitable regulatory sequence which allows expression, i.e. transcription and translation of the inserted isolated and purified DNA sequence.

As used herein, the term “promoter” designates any additional regulatory sequences as known in the art e.g. a promoter and/or an enhancer, polyadenylation sites and splice junctions usually employed for the expression of the polypeptide or may include additionally one or more separate targeting sequences and may optionally encode a selectable marker. Promoters which can be used provided that such promoters are compatible with the host cell are e.g. promoters obtained from the genomes of viruses such as polyoma virus, adenovirus (such as Adenovirus 2), papilloma virus (such as bovine papilloma virus), avian sarcoma virus, cytomegalovirus (such as murine or human cytomegalovirus immediate early promoter), a retrovirus, hepatitis-B virus, and Simian Virus 40 (such as SV 40 early and late promoters) or promoters obtained from heterologous mammalian promoters, such as the actin promoter or an immunoglobulin promoter or heat shock promoters.

Enhancers which can be used are e.g. enhancer sequences known from mammalian genes (globin, elastase, albumin, a-fetoprotein, and insulin) or enhancer from a eukaryotic cell virus. e.g. the SV40 enhancer, the cytomegalovirus early promoter enhancer, the polyoma, and adenovirus enhancers.

A wide variety of host/expression vector combinations may be employed in expressing the DNA sequences of this invention. Useful expression vectors, for example, may consist of segments of chromosomal, non-chromosomal and synthetic DNA sequences. Suitable vectors include derivatives of SV40 and known bacterial plasmids, e.g., E. coli plasmids col El, pCR1, pBR322, pcDNA3, pMB9 and their derivatives, plasmids such as RP4; phage DNAs, e.g., the numerous derivatives of phage X, e.g., NM989, and other phage DNA, e.g., M13 and filamentous single stranded phage DNA; yeast plasmids such as the 2μ plasmid or derivatives thereof; vectors useful in eukaryotic cells, such as vectors useful in insect or mammalian cells; vectors derived from combinations of plasmids and phage DNAs, such as plasmids that have been modified to employ phage DNA or other expression control sequences; and the like. Most preferably the expression vector is pcDNA3.

Another concern of the present invention is to provide a eukaryotic or prokaryotic host cell containing the peptide according to the invention, the isolated and purified nucleic acid sequence of the invention or and/or expression vector described herein.

Transformation or transfection of appropriate eukaryotic or prokaryotic host cells with an expression vector comprising a purified and isolated DNA sequence according to the invention is accomplished by well known methods that typically depend on the type of vector used. With regard to these methods, see for example, Maniatis et al. 1982, Molecular Cloning, A laboratory Manual, Cold Spring Harbor Laboratory and commercially available methods. The term “cell transfected” or “cell transformed” or “transfected/transformed cell” means the cell into which the extracellular DNA has been introduced and thus harbours the extracellular DNA. The DNA might be introduced into the cell so that the nucleic acid is replicable either as a chromosomal integrant or as an extra chromosomal element.

The peptide consisting essentially of the N2 sequence of the RasGAP protein, a fragment thereof, or a variant thereof, optionally conjugated to an agent which increases the accumulation of the peptide in a cell as described herein are preferably produced, recombinantly, in a cell expression system. A wide variety of unicellular host cells are useful in expressing the DNA sequences of this invention. These hosts may include well known eukaryotic and prokaryotic hosts, such as strains of E. coli, Pseudomonas, Bacillus, Streptomyces, fungi such as yeasts, and animal cells, such as CHO, YB/20, NSO, SP2/0, R1.1, B-W and L-M cells, African Green Monkey kidney cells (e.g., COS 1, COS 7, BSC1, BSC40, and BMT10), insect cells (e.g., Sf9), and human cells and plant cells in tissue culture. Preferably, the host cell is a bacterial cell, more preferably an E. coli cell.

Usually the medicament of the invention comprises a pharmaceutically effective amount of the peptide of the invention. “A pharmaceutically effective amount” refers to a chemical material or compound which, when administered to a human or animal organism induces a detectable pharmacologic and/or physiologic effect, i.e. to enhance the ability of a radiation therapy to kill cancer cells or to enhance the effect of said radiation therapy on tumors.

The respective pharmaceutically effect amount can depend on the specific patient to be treated, on the disease to be treated and on the method of administration. Further, the pharmaceutically effective amount depends on the specific peptide used. The treatment usually comprises a multiple administration of the pharmaceutical composition, usually in intervals of several hours, days or weeks. The pharmaceutically effective amount of a dosage unit of the peptide of the invention usually is in the range of 0.001 ng to 1000 mg per kg of body weight of the patient to be treated.

Preferably, in addition to at least one peptide as described herein, the pharmaceutical composition may contain one or more pharmaceutically acceptable carriers, diluents and adjuvants. Acceptable carriers, diluents and adjuvants which facilitates processing of the active compounds into preparation which can be used pharmaceutically are non-toxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl orbenzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN®, PLURONICS® or polyethylene glycol (PEG).

The form of administration of the pharmaceutical composition may be systemic or topical. For example, administration of such a composition may be various parenteral routes such as subcutaneous, intravenous, intradermal, intramuscular, intraperitoneal, intranasal, transdermal, buccal routes or via an implanted device, and may also be delivered by peristaltic means.

The pharmaceutical composition comprising a peptide, as described herein, as an active agent may also be incorporated or impregnated into a bioabsorbable matrix, with the matrix being administered in the form of a suspension of matrix, a gel or a solid support. In addition the matrix may be comprised of a biopolymer.

Sustained-release preparations may be prepared. Suitable examples of sustained-release preparations include semi permeable matrices of solid hydrophobic polymers containing the antibody, which matrices are in the form of shaped articles, e.g. films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and [gamma]ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid.

The formulations to be used for in vivo administration must be sterile. This is readily accomplished for example by filtration through sterile filtration membranes.

Alternatively also, the medicament is administered, prior to, during and/or after said patient was subjected to radiotherapy and chemotherapy.

It is understood that the suitable dosage of a peptide of the present invention will be dependent upon the age, sex, health, and weight of the recipient, kind of concurrent treatment, if any and the nature of the effect desired.

The appropriate dosage form, as well as the duration of the administration, will depend on the disease, the peptide, and the mode of administration; possibilities include tablets, capsules, lozenges, dental pastes, suppositories, inhalants, solutions, ointments and parenteral depots.

Since amino acid modifications of the amino acids of the peptide are also encompassed in the present invention, this may be useful for cross-linking the peptide of the invention to a water-insoluble matrix or the other macromolecular carriers, or to improve the solubility, adsorption, and permeability across the blood brain barrier. Such modifications are well known in the art and may alternatively eliminate or attenuate any possible undesirable side effect of the peptide and the like.

While a preferred pharmaceutical composition of the present invention comprises a peptide as an active agent, an alternative pharmaceutical composition may contain a purified and isolated nucleic acid sequence encoding the peptide, as described herein, as an active agent. This pharmaceutical composition may include either the sole purified and isolated DNA sequence, an expression vector comprising said purified and isolated DNA sequence or a host cell previously transfected or transformed with an expression vector described herein. In this latter example, host cell will preferably be isolated from the patient to be treated in order to avoid any antigenicity problem. These gene and cell therapy approaches are especially well suited for patients requiring repeated administration of the pharmaceutical composition, since the said purified and isolated DNA sequence, expression vector or host cell previously transfected or transformed with an expression vector can be incorporated into the patient's cell which will then produce the protein endogenously.

“Administering”, as it applies in the present invention, refers to contact of the pharmaceutical composition to the subject, preferably a human.

For systemic administration, a therapeutically effective amount or dose can be estimated initially from in vitro assays. For example, a dose can be formulated in animal models to achieve a circulating concentration range that includes the IC50 as determined in cell culture. Such information can be used to more accurately determine useful doses in humans.

Initial doses can also be estimated from in vivo data, e.g. animal models, using techniques that are well known in the art. One ordinarily skill in the art could readily optimise administration to humans based on animal data and will, of course, depend on the subject being treated, on the subject's weight, the severity of the disorder, the manner of administration and the judgement of the prescribing physician.

Since radiation therapy can alternatively be used to treat cancer in combination with chemotherapy, the present invention also comprises administering the medicament prior to, during and/or after said patient was subjected to a radiation therapy and chemotherapy.

The present disclosure also provides a method of treatment of cancer and/or tumors comprising administering to a patient in need thereof, a therapeutically effective amount of

i) a peptide consisting essentially of the N2 sequence of the RasGAP protein, a biologically active fragment thereof, or a variant thereof, or
ii) a peptide consisting essentially of the N2 sequence of the RasGAP protein, a biologically active fragment thereof, or a variant thereof, conjugated to an agent which increases the accumulation of said peptide in a cell,
prior to, during and/or after said patient was subjected to a radiation therapy and so that said peptide enhances the ability of said radiation therapy to kill cancer cells or reduce tumors.

As used herein, the term “therapeutically effective amount” of the peptide of the invention, with respect to the subject method of treatment, refers to an amount of the peptide of the invention which, when administered prior to, during and/or after the patient was subjected to a radiation therapy, as part of desired dose regimen, enhances the properties of said radiation therapy, e.g. a change in the rate of cell proliferation and/or state of differentiation and/or rate of survival of a cell to clinically acceptable standards. This amount may further relieve to some extent one or more of the symptoms of a neoplasia disorder, including, but is not limited to: 1) reduction in the number of cancer cells; 2) reduction in tumor size; 3) inhibition (i.e., slowing to some extent, preferably stopping) of cancer cell infiltration into peripheral organs; 4) inhibition (i.e., slowing to some extent, preferably stopping) of tumor metastasis; 5) inhibition, to some extent, of tumor growth; 6) relieving or reducing to some extent one or more of the symptoms associated with the disorder; and/or 7) relieving or reducing the side effects associated with the administration of anticancer therapies.

In preferred methods, the subject is a human patient, and the administered peptide is the TAT-RasGAP₃₁₇₋₃₂₆ peptide or R9-RasGAP₃₁₇₋₃₂₆ peptide. Most preferably, these peptides are in the retro-inverso (RI) form as described herein.

Since radiation therapy can alternatively be used to treat cancer in combination with chemotherapy, the peptide and the method of the invention further comprise administering a chemotherapy treatment, prior to, during and/or after said patient was subjected to a radiation therapy.

Also, since the most significant cause for treatment failure and cancer mortality is radio resistance, another embodiment envisioned that the peptide and the method of the invention are used in case the cancer (or parts of it, as in hypoxic parts) of the patient to be treated is radio resistant.

Embraced by the scope of the present invention is also an in vivo method of enhancing the ability of a radiation therapy to kill cancer cells or to enhance the effect of said radiation therapy on tumors comprising contacting a cancer cell with at least one peptide consisting essentially of the N2 sequence of the RasGAP protein, a fragment thereof, or a variant thereof, conjugated or not to an agent which increases the accumulation of said peptide in said cell prior to, during and/or after said cancer cell was subjected to a radiation therapy.

Also embraced in the scope of the invention is an in vivo method of sensitizing cancer cells to radiation therapy comprising contacting a cancer cell with at least one peptide consisting essentially of the N2 sequence of the RasGAP protein, a fragment thereof, or a variant thereof, conjugated or not to an agent which increases the accumulation of said peptide in said cell prior to, during and/or after said cancer cell was subjected to a radiation therapy.

The invention further comprises a kit for treating or cancer in a subject, said kit comprising at least one peptide consisting essentially of the N2 sequence of the RasGAP protein, a fragment thereof, or a variant thereof, conjugated or not to an agent which increases the accumulation of said peptide in said cell, optionally with reagents and/or instructions for use.

Those skilled in the art will appreciate that the invention described herein is susceptible to variations and modifications other than those specifically described. It is to be understood that the invention includes all such variations and modifications without departing from the spirit or essential characteristics thereof. The invention also includes all of the steps, features, compositions and compounds referred to or indicated in this specification, individually or collectively, and any and all combinations or any two or more of said steps or features. The present disclosure is therefore to be considered as in all aspects illustrated and not restrictive, the scope of the invention being indicated by the appended Claims, and all changes which come within the meaning and range of equivalency are intended to be embraced therein.

Various references are cited throughout this Specification, each of which is incorporated herein by reference in its entirety.
The foregoing description will be more fully understood with reference to the following Examples. Such Examples, are, however, exemplary of methods of practising the present invention and are not intended to limit the scope of the invention.

### EXAMPLE

### Example 1

### The Radio-Sensitization Effect of TAT-RasGAP317-326 is Independent of the p53 Status of Cancer Cells

### Clonogenic Assay

One hundred HeLa cells or 500 HCT116p53^{+/+} or 500 HCT116p53^{-/-} cells were seeded in 8.5 cm plates and after 24 hours later treated or not with TAT or TAT-RasGAP317-326 (20 μM) prior exposure to the various doses of γ radiation (0, 1, 2, 4, 6 or 8 Gray).

After two weeks, the culture medium was removed and the plates were gently washed with water. A crystal violet solution (0.5% crystal violet in 25% methanol) was then added to stain colonies and gently washed out with water. The number of colonies was then counted.

### Results

Data shown in FIG. 1 indicate that TAT-RasGAP317-326 increases the efficacy of γ-irradiation-mediated cell killing in different tumor cell lines (HCT116p53^{+/+} and HCT116p53^{-/-}), but not in a non-cancer cell line (HaCat), as assessed by the clonogenic assay.

Furthermore, in contrast to what has been reported before for the genotoxin-sensitization on tumor growth (Michod D, Widmann C, 2007, see above), the peptide of the invention does not require a functional p53 cellular status to increase the sensitivity of cancer and tumor cells to radiotherapy. Indeed, FIG. 1A-B shows that TAT-RasGAP317-326 increases the efficacy of γ-irradiation on tumor cells, regardless of whether the tumor cells bear a functional p53 gene (HCT116 p53^{+/+}) or not (HCT116 p53^{-/-}).

### Example 2

### The Radio-Sensitization Effect of TAT-RasGAP317-326 is Specific for Cancer Cell Lines and Does not Operate in Non-Cancer Cell Lines

The clonogenic formation assay (CFA) was performed using cancer cell lines (HCT116 with or without p53) (FIG. 1A-B) and non-tumorigenic HaCaT cells (FIG. 1C) that were subjected to the various doses of γ radiation (0, 1, 2, or 4, Gray), in the presence or in absence of TAT-RasGAP317-326 (20 μM). The number of colonies was determined two weeks after irradiation.

### Results

TAT-RasGAP317-326 has no influence on the γ-radiation-sensitivity of HaCAT cells (FIG. 1C), a non-tumor keratinocyte-derived cell line whereas it increases the efficacy of γ-irradiation-mediated cell killing in the HCT116 cell lines (FIG. 1A-B).

### Example 3

### Radio-Sensitization Effect of TAT-RasGAP317-326 in Mice Bearing HCT116 Xenografts

Immunodeficient mice bearing subcutaneous tumors allow easy access of the tumors to defined fractionated irradiation doses with minimal effect on surrounding tissues and without the need of anesthesia. This latter point is important because anesthesia can modify the response to radiation therapy (Denekamp, 1979) and anesthesia is generally not used in patients during radiotherapy.

HCT116 tumor cells, lacking or not p53, were grafted onto the back of female Swiss homozygous nu/nu mice leading to the development of subcutaneous tumors. Then, the immunodeficient mice bearing subcutaneous tumors were divided into four experimental groups as follows:

1) Intraperitoneal PBS injections and no irradiation.
2) Intraperitoneal TAT-RasGAP317-326 injections (every day, 1 mg peptide per kg of mouse in 300 μl PBS) and no irradiation.
3) Intraperitoneal PBS injection and γ-irradiation (3 Gy).
4) Intraperitoneal TAT-RasGAP317-326 injections and γ-irradiation.

The treatments (i.e. peptide injections followed by irradiation) were performed every day for 10 days without interruption. γ-irradiation was performed 1 hour after the peptide injection. The size of the tumors was monitored 3 times a week. #: mice were sacrificed when the tumors reached a volume of about 800 mm³.

### Results

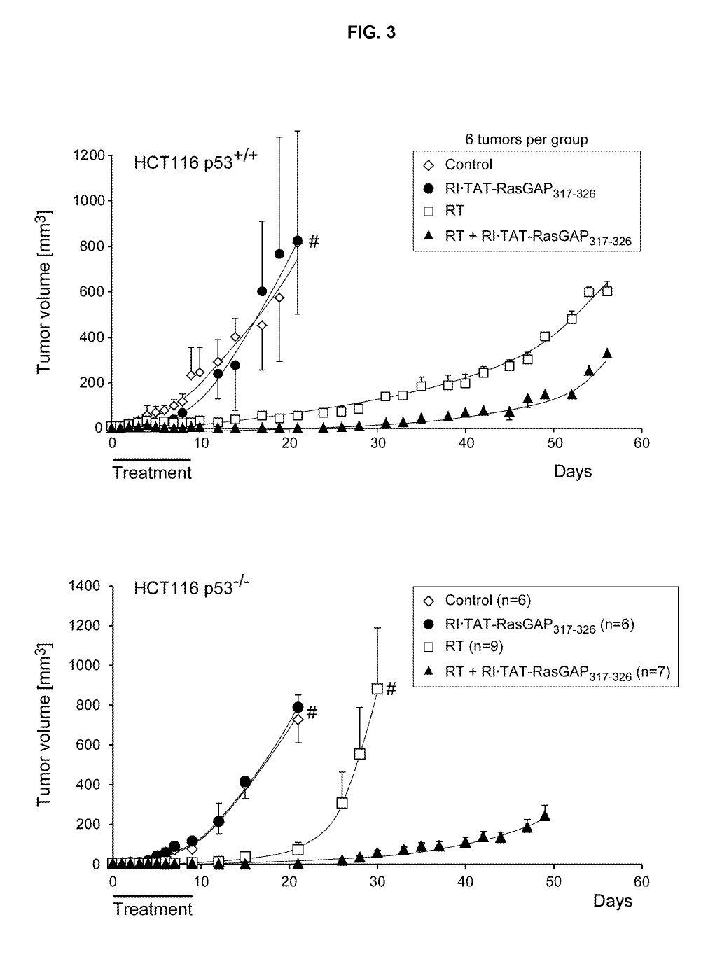

The experiment performed with this model shows a sensitizing effect of TAT-RasGAP317-326 on irradiated tumors leading to significant growth delay and reduction in tumor volume (FIG. 3). The radiotherapy-sensitization ability of the peptide did not require a functional p53 status in the tumors as the growth of HCT116 p53^{-/-} tumors was very efficiently hampered by the peptide (FIG. 3, lower graph).

1.-19. (canceled)

20. A method of cancer and/or tumors comprising administering to a patient in need thereof, a therapeutically effective amount of
i) a peptide consisting essentially of the N2 sequence of the RasGAP protein, a biologically active fragment thereof, or a variant thereof, or
ii) a peptide consisting essentially of the N2 sequence of the RasGAP protein, a biologically active fragment thereof, or a variant thereof, conjugated to an agent which increases the accumulation of said peptide in a cell, prior to, during and/or after said patient was subjected to a radiation therapy wherein said peptide enhances the ability of said radiation therapy to kill cancer cells or reduce tumors.

21. The method according to claim 20, wherein the radiation therapy is either external radiation therapy or internal radiation therapy.

22. The method according to claim 21, wherein the source for external radiation therapy is selected from the group consisting of x-rays, gamma rays and particle beams; or a combination thereof.

23. The method according to claim 21, wherein the source for internal radiation therapy is selected from the group consisting of radioactive iodine; strontium89; and a radioisotope of phosphorous, palladium, cesium, indium, phosphate, or cobalt; or a combination thereof.

24. The method according to claim 20, wherein the biologically active fragment of the N2 sequence of the RasGAP protein comprises a complete or a part of the amino acid sequence of the SH3 domain, or a variant thereof.

25. The method according to claim 24, wherein the biologically active fragment comprising a complete or a part of the amino acid sequence of the SH3 domain, or the variant thereof, contains less than or equal to 70 amino acids of the amino acid sequence of the SH3 domain.

26. The method according to claim 20, wherein the biologically active fragment comprising a complete or a part of the amino acid sequence of the SH3 domain is selected from the group consisting of SEQ ID No. 14, SEQ ID No. 15, SEQ ID No. 16 and SEQ ID No. 5; or a variant thereof.

27. The method according to claim 20, wherein the variant of the N2 sequence of the RasGAP protein, or of a biologically active fragment thereof, comprises the general amino acid sequence WXWVTXXRTX (SEQ ID No. 13), wherein X represents an amino acid.

28. The method according to claim 27, wherein the variant of the N2 sequence of the RasGAP protein, or of a biologically active fragment thereof, comprises an amino acid sequence selected from the group consisting of WLWVTAHRTG (SEQ ID No. 9), WLWVSNLRTD (SEQ ID No. 11) and WMWVTNHRTD (SEQ ID No. 12).

29. The method according to claim 20, wherein the biologically active fragment comprising a complete or a part of the amino acid sequence of the SH3 domain is encoded by a DNA sequence selected from the group consisting of SEQ ID No. 1, SEQ ID No. 2, SEQ ID No. 3 and SEQ ID No. 4.

30. The method according to claim 20, wherein the agent which increases the accumulation of said peptide in a cell is a cell membrane permeable carrier.

31. The method according to claim 30, wherein the cell membrane permeable carrier is a peptide.

32. The method according to claim 31, wherein the cell membrane permeable carrier peptide is a positively charged amino acid rich peptide.

33. The method according to claim 32, wherein the positively charged amino acid rich peptide is an arginine rich peptide selected from the group consisting of HIV-TAT 48-57 peptide, FHV-coat₃₅₋₄₉ peptide, HTLV-II Rex₄₋₁₆ peptide, BMV gag₇₋₂₅ peptide and R9 peptide.

34. The method according to claim 33, wherein the peptide consisting essentially of the N2 sequence of the RasGAP protein, a biologically active fragment thereof, or a variant thereof is either in the L-form or in D-form and/or in a retro-inverso isomer form.

35. The method according to claim 20, wherein the agent which increases the accumulation of the peptide consisting essentially of the N2 sequence of the RasGAP protein, a biologically active fragment thereof, or a variant thereof, is either in the L-form or in D-form and/or in a retro-inverso isomer form.

36. The method according to claim 20, wherein the therapeutically effective amount of the peptide is administered prior to, during, and/or after said patient was subjected to chemotherapy.

37. An in vivo method of sensitizing cancer cells to radiation therapy in a patient in need thereof, said method comprising contacting a cell with at least one peptide consisting essentially of the N2 sequence of the RasGAP protein, a biologically active fragment thereof, or a variant thereof, conjugated or not to an agent which increases the accumulation of said peptide in said cell.

38. An in vivo method of enhancing radiation therapy in a patient in need thereof, said method comprising contacting a cell with at least one peptide consisting essentially of the N2 sequence of the RasGAP protein, a biologically active fragment thereof, or a variant thereof, conjugated or not to an agent which increases the accumulation of said peptide in said cell wherein said peptide is contacted prior to, during, and/or after said patient was subjected to a radiation therapy.


SEQUENCE LISTING (28 sequences)

SEQ ID NO: 1 - DNA, 249 bp, Homo sapiens
gaagatagaaggcgtgtacgagctattctaccttacacaaaagtaccagacactgatgaaataagtttcttaaaaggagatatgttcattgttcataatgaattagaagatggatggatgtgggttacaaatttaagaacagatgaacaaggccttattgttgaagacctagtagaagaggtgggccgggaagaagatccacatgaaggaaaaatatggttccatgggaagatttccaaacaggaagct

SEQ ID NO: 2 - DNA, 204 bp, Homo sapiens
gtacgagctattctaccttacacaaaagtaccagacactgatgaaataagtttcttaaaaggagatatgttcattgttcataatgaattagaagatggatggatgtgggttacaaatttaagaacagatgaacaaggccttattgttgaagacctagtagaagaggtgggccgggaagaagatccacatgaaggaaaaatatgg

SEQ ID NO: 3 - DNA, 174 bp, Homo sapiens
gtacgagctattctaccttacacaaaagtaccagacactgatgaaataagtttcttaaaaggagatatgttcattgttcataatgaattagaagatggatggatgtgggttacaaatttaagaacagatgaacaaggccttattgttgaagacctagtagaagaggtgggccgg

SEQ ID NO: 4 - DNA, 30 bp, Homo sapiens
tggatgtgggttacaaatttaagaacagat

SEQ ID NO: 5 - protein, 10 aa, Homo sapiens
WMWVTNLRTD

SEQ ID NO: 6 - protein, 298 aa, Homo sapiens
SLDGPEYEEEEVAIPLTAPPTNQWYHGKLDRTIAEERLRQAGKSGSYLIRESDRRPGSFVLSFLSQMNVVNHFRIIAMCGDYYIGGRRFSSLSDLIGYYSHVSCLLKGEKLLYPVAPPEPVEDRRRVRAILPYTKVPDTDEISFLKGDMFIVHNELEDGWMWVTNLRTDEQGLIVEDLVEEVGREEDPHEGKIWFHGKISKQEAYNLLMTVGQVCSFLVRPSDNTPGDYSLYFRTNENIQRFKICPTPNNQFMMGGRYYNSIGDIIDHYRKEQIVEGYYLKEPVPMQDQEQVLNDTVD

SEQ ID NO: 7 - protein, 22 aa, Homo sapiens
DTRLNTVWMWGGRRRQRRKKRG

SEQ ID NO: 8 - protein, 10 aa, Homo sapiens
DTRLNTVWMW

SEQ ID NO: 9 - protein, 10 aa, Anopheles sp.
WLWVTAHRTG

SEQ ID NO: 10 - protein, 10 aa, Anopheles sp.
GTRHATVWLW

SEQ ID NO: 11 - protein, 10 aa, Artificial Sequence
  synthetic peptide
WLWVSNLRTD

SEQ ID NO: 12 - protein, 10 aa, Artificial Sequence
  synthetic peptide
WMWVTNHRTD

SEQ ID NO: 13 - protein, 10 aa, Artificial Sequence
  synthetic peptide
WXWVTXXRTX

SEQ ID NO: 14 - protein, 83 aa, Homo sapiens
EDRRRVRAILPYTKVPDTDEISFLKGDMFIVHNELEDGWMWVTNLRTDEQGLIVEDLVEEVGREEDPHEGKIWFHGKISKQEA

SEQ ID NO: 15 - protein, 69 aa, Homo sapiens
RVRAILPYTKVPDTDEISFLKGDMFIVHNELEDGWMWVTNLRTDEQGLIVEDLVEEVGREEDPHEGKIW

SEQ ID NO: 16 - protein, 59 aa, Homo sapiens
RVRAILPYTKVPDTDEISFLKGDMFIVHNELEDGWMWVTNLRTDEQGLIVEDLVEEVGR

SEQ ID NO: 17 - protein, 10 aa, Artificial Sequence
  synthetic peptide
DTRLNSVWLW

SEQ ID NO: 18 - protein, 10 aa, Artificial Sequence
  synthetic peptide
DTRHNTVWMW

SEQ ID NO: 19 - protein, 10 aa, Artificial Sequence
  synthetic peptide
XTRXXTVWXW

SEQ ID NO: 20 - protein, 22 aa, Artificial Sequence
  synthetic peptide
GTRHATVWLWGGRRRQRRKKRG

SEQ ID NO: 21 - protein, 22 aa, Artificial Sequence
  synthetic peptide
DTRLNSVWLWGGRRRQRRKKRG

SEQ ID NO: 22 - protein, 22 aa, Artificial Sequence
  synthetic peptide
DTRHNTVWMWGGRRRQRRKKRG

SEQ ID NO: 23 - protein, 22 aa, Artificial Sequence
  synthetic peptide
XTRXXTVWXWGGRRRQRRKKRG

SEQ ID NO: 24 - protein, 21 aa, Artificial Sequence
  synthetic peptide
DTRLNTVWMWGGRRRRRRRRR

SEQ ID NO: 25 - protein, 21 aa, Artificial Sequence
  synthetic peptide
GTRHATVWLWGGRRRRRRRRR

SEQ ID NO: 26 - protein, 21 aa, Artificial Sequence
  synthetic peptide
DTRLNTVWMWGGRRRRRRRRR

SEQ ID NO: 27 - protein, 21 aa, Artificial Sequence
  synthetic peptide
DTRHNTVWMWGGRRRRRRRRR

SEQ ID NO: 28 - protein, 21 aa, Artificial Sequence
  synthetic peptide
XTRXXTVWXWGGRRRRRRRRR